(19) United States
(12) Patent Application Publication
(10) Pub. No.: US 2026/0341397 A1
(43) Pub. Date: Oct. 29, 2026

(54) MODELING PROTEIN CONFORMATIONAL ENSEMBLES USING SPECTROSCOPIC DISTANCE CONSTRAINTS

(71) Applicants: The Medical College of Wisconsin, Inc., Milwaukee, WI (US); Duke University, Durham, NC (US); Georgia Institute of Technology, Atlanta, GA (US)

(72) Inventors: Michael Taylor Lerch, St. Francis, WI (US); Julian David Grosskopf, Milwaukee, WI (US); Biswaranjan Pani, Durham, NC (US); Peter Kasson, Atlanta, GA (US)

(21) Appl. No.: 19/659,699

(22) Filed: Apr. 27, 2026

Related U.S. Application Data
(60) Provisional application No. 63/795,085, filed on Apr. 25, 2025.

(51) Int. Cl. G16B 15/20; G06N 20/20; G16B 15/30; G16B 40/10

(52) U.S. Cl. CPC G16B 15/20; G06N 20/20; G16B 15/30; G16B 40/10

(57) ABSTRACT

A method for modeling protein conformational ensembles using distance constraints is provided herein. The method includes obtaining experimental distance distribution data for a protein, generating an initial protein structural model, and iteratively refining the protein structural model. The iterative refinement includes simulating a distance distribution for the current model using in silico spin labeling, comparing the simulated distance distribution to the experimental distance distribution data, calculating a distance update metric based on differences between the simulated and experimental distributions, and applying a bias potential in a molecular dynamics simulation to drive the protein structure toward target distances updated by the distance update metric. The method further includes generating an ensemble of refined protein structural models that are consistent with the experimental distance distribution data.

### CROSS-REFERENCE TO RELATED APPLICATIONS

This application claims the benefit of U.S. Provisional Patent Application Ser. No. 63/795,085, filed on Apr. 25, 2025, and entitled “MODELING PROTEIN CONFORMATIONAL ENSEMBLES USING SPECTROSCOPIC DISTANCE CONSTRAINTS,” which is herein incorporated by reference in its entirety.

### STATEMENT OF FEDERALLY SPONSORED RESEARCH

This invention was made with government support under R01 GM135581, and R01 GM138444 awarded by the National Institutes of Health. The government has certain rights in the invention.

### BACKGROUND

Protein conformational dynamics play an important role in biological processes, including signal transduction, enzyme catalysis, and drug-receptor interactions. Understanding these dynamic structural changes is advantageous for elucidating protein function and developing targeted therapeutics. However, capturing the full range of conformational states adopted by proteins remains challenging due to limitations in traditional structural biology techniques.

Spectroscopic methods such as double electron-electron resonance (DEER) can provide valuable distance constraint information to probe protein conformational ensembles. Integrating this experimental data with computational modeling approaches offers the potential to generate more complete representations of protein conformational states and structural dynamics. Nonetheless, accurately incorporating multiple distance constraints into molecular simulations while maintaining physically realistic protein conformations presents significant technical hurdles. Improved methods for modeling protein conformational ensembles that faithfully reproduce experimental distance measurements could advance the understanding of protein function and aid structure-based drug design efforts.

### SUMMARY OF THE DISCLOSURE

According to an aspect of the present disclosure, a method for modeling protein conformational ensembles using distance constraints is provided. The method includes obtaining experimental distance distribution data for a protein, generating an initial protein structural model, iteratively refining the protein structural model, and generating an ensemble of refined protein structural models consistent with the experimental distance distribution data. The iterative refinement includes simulating a distance distribution for the current model using in silico spin labeling, comparing the simulated distance distribution to the experimental distance distribution data, calculating a distance update metric based on differences between the simulated distance distribution and the experimental distance distribution, and applying a bias potential in a molecular dynamics simulation to drive the protein structure toward target distances updated by the distance update metric.

### BRIEF DESCRIPTION OF THE DRAWINGS

FIGS. 1A-1C illustrate a schematic of the modeling framework according to some embodiments described in the present disclosure. In an update block (FIG. 1A) of the modeling framework, the prior output model is used as an input, and an update to the target distance between the Ca atoms of the spin labeled residues is calculated based on the space within the experimental data not explored by the prior model. The updated Cα-Cα distance targets serve as constraints for the next modelling iteration. In a biased resampling ensemble refinement (BRER) block (FIG. 1B) of the modeling framework, the distance update is driven by a learned linear bias potential applied globally and not restricted to the label sites. When incorporating datasets encompassing multiple spin pairs, the bias potential as a function of each pair is optimized simultaneously to allow for coordinated conformational rearrangements. In an analysis block (FIG. 1C) of the modeling framework, the models generated at each iteration of the BRER block are binned, ranked, and a representative subset is selected for further structural and statistical analysis.

FIG. 2 is a flowchart of an example method for modeling protein conformational ensembles using distance constraints obtained from spectroscopic measurements.

FIGS. 3A-3C illustrate spin-label rotameric heterogeneity. FIG. 3A shows rotamers of the spin-label side chain IAP (named for the spin label reagent 3-(2-Iodoacetamido)-2,2,5,5-tetramethyl-1-pyrrolidinyloxy) (green) modeled on to the β2AR (grey cartoon, PDB ID: 3SN6, loops modelled in), shown from the cytoplasmic face. Transparent clouds represent the nitroxide N—O atoms—the location of the free radical—which ultimately serves as the point of measurement in DEER spectroscopy. Orange spheres represent the C_{α} atoms of the spin-labeled residues, with an orange dashed line signifying the distance between C_{α} atoms. FIG. 3B shows a close up on the IAP spin label, with a single rotamer highlighted. FIG. 3C is a graph of the IAP nitroxide distribution (green) and distance between C_{α} atoms of the IAP-labeled residues (orange dotted line).

FIGS. 4A-4F illustrate an example of modeling the outward active conformation of TM6 in the β2AR using a single IAP spin label pair at 148/266. FIG. 4A: Target Cα distance of the biased MD simulations vs. the Cα distance of the production phase trajectory average of each modelling iteration. Data points (grey dots) are fit to a line (black) with one standard deviation in the fitting parameters (grey shade). A perfectly matched correlation between the trajectory average and the target distance would yield a line with slope 1 and intercept 0, modelled as a red line. FIG. 4B: Graph of the simulated distribution of the initial model (grey), the experimental data (blue), the simulated distributions of the top 10 models (multi-colored lines), and the sum of the top 10 models (black line). All distributions are area normalized. FIGS. 4C-4F: Cartoon representations of the starting model (grey), the crystal structure of active β2AR (PDB ID: 3P0G), a representative model (magenta) from the top 10 models, and the top ten models shown as transparent cartoons (multi-colored). Residues N148 (TM4) and L266 are shown as sticks. Intracellular loop (ICL) 3 has been truncated in all models shown here for visual purposes.

FIG. 5 shows Cα RMSF of the top 10 β2AR models. The grey-shaded region indicates residues 261-280 of TM6.

FIGS. 6A-6C show results from modelling NMF components of the ATIR from the data in Wingler L M, et al., “Angiotensin Analogs with Divergent Bias Stabilize Distinct Receptor Conformations,” Cell. 2019; 176 (3): 468-478. FIG. 6A: Graphs depicting the NMF conformation/input data (blue) vs. the sum of the simulated spin label distributions of the top 50 models (colored). Starting model simulated distributions are represented in grey. FIG. 6B: Representative ATIR conformational models (color) overlayed with the starting model generated from an unbiased MD equilibration of the antagonist bound ATIR structure (PDB ID: 4YAY). Each model was selected from the top 5 models ranked by their closeness to the NMF component data. Label sites are shown as their native residues represented by sticks. Arrows indicate major structural changes observed in the overlayed structures. FIG. 6C: Ensemble of the top 5 models in each NMF conformation. Models highlighted with a black silhouette are the models representing the conformational components in panel B.

FIG. 7 shows an RMSF plot of top 50 models for each ATIR component. Dashed black lines represent Cα atoms of spin label sites with the specific site labeled next to each line.

FIGS. 8A-8E show model and motif analysis of the ATIR models generated from NMF conformations. FIG. 8A: PCA of ATIR model backbone atom coordinates (upper) and ATIR model label site Cα atom coordinates (lower). FIG. 8B: Analysis of the L70-P299 (sticks) Cα distance tracking movement of the NPxxY motif. Representative models of conformations 2 (orange) and 3 (magenta) are shown as cartoons, and the distance (Å) between the Cα atoms are highlighted by dashed lines. For each NMF conformation, the distance between Cα atoms of L70 and P299 were analyzed and plotted as a box and whisker plot where the median value is indicated by the center line, lower and upper bounds indicate the first and third quartile values, the upper and lower bounds of the whiskers indicate the furthest datapoint lying within −1.5 IQR and +1.5 IQR, respectively, the black dots represent data points, the open circles represent outliers, and the stars are the values of Cα distance of the models in the upper panel. The dotted line indicates the cutoff of L70 and P299 Cα distance describing B arrestin bias. FIGS. 8C-8E: Comparison of the conformation 1 model to conformation 2 (FIG. 8C), conformation 3 (FIG. 8D), and conformation 4 (FIG. 8E). Models are shown as cartoons, native label site residues are shown as sticks, and relevant conformational rearrangements are highlighted with a black arrow.

FIGS. 9A-9E show NPxxY and DRY motif changes consistent with published X-ray crystallography and cryo-EM structures. FIG. 9A shows L70-P299 Cα atom distance as a monitor of NPxxY movement of the ATIR models. Representative models of conformations 2 (orange) and 3 (green) are shown as cartoons, and the distance between the Cα atoms are highlighted by dashed lines. FIG. 9B shows box and whisker plot for L70-P299 Cα atom distance of models generated in each conformation. The median value is indicated by the center line and lower and upper bounds indicate the first and third quartile values. Upper and lower bounds of the whiskers indicate the furthest datapoint lying within-1.5 IQR and +1.5 IQR, respectively. Black dots represent data points, open circles represent outliers, and the stars are the values of the models in FIG. 9A. The black dashed line at 7 Å represents the upper limited considered for β arrestin biased conformations of the NPXXY measured by L70-P299 distance. FIG. 9C shows box and whisker plot of the distance between D125 Cγ and R126 Cζ of the DRY motif for the models in each component. The median value is indicated by the center line and lower and upper bounds indicate the first and third quartile values. Upper and lower bounds of the whiskers indicate the furthest datapoint lying within −1.5 IQR and +1.5 IQR, respectively. Black dots represent data points, open circles represent outliers, and the stars are the values of the models in FIGS. 9D and 9E. The black dotted line is the distance between D125 Cγ and R126 Cζ atoms of 60S1. FIG. 9D shows an overlay of the TRV023 (β arrestin biased ligand) bound ATIR X-ray crystal structure (PDBID: 6OS1) with a representative model of conformation 3. Blow up panels show the DRY motif of each structure/model. A salt bridge interaction between D125 and R126 is shown by the yellow dashed line. FIG. 9e shows an overlay of the ATIR-Gq complex bound to Sar1-AngII (PDBID: 7F6G) with a representative model of component 4. The α5 helix of the Gq protein is shown in pale cyan. The upper panel shows R126 of the ATIR interacting with Y356 of the Gq α5 helix, shown by a yellow dashed line.

FIG. 10 is a block diagram of an example system for modeling protein conformational ensembles using a spectroscopic distance constraint.

FIG. 11 is a block diagram of example components that can implement the system of FIG. 10.

### DETAILED DESCRIPTION

Described here are systems and methods for modeling conformations of G protein coupled receptors (GPCRs), or other macromolecular signaling assemblies, using spectroscopic data. The systems and methods described herein are capable of generating structural models of GPCR conformations based on experimental spectroscopic data, such as continuous-wave electron paramagnetic resonance (CW EPR) spectroscopy data, double electron-electron resonance (DEER) data, and the like. This allows for the exploration of conformational states that may not be readily observable using traditional structural biology techniques.

By combining computational modeling techniques with experimental constraints derived from spectroscopic data, the disclosed systems and methods enable the generation of atomic-level structural models that represent different functional states of GPCRs, other drug pathological protein complexes, or other macromolecular signaling assemblies. By way of example, these models may provide insights into the mechanisms of GPCR activation, ligand binding, and signal transduction, including biased signaling.

The systems and methods described herein may have applications in drug discovery and development, particularly in the design of ligands with specific efficacy profiles or biased signaling properties. By providing a more comprehensive understanding of GPCR conformational ensembles, these approaches may facilitate the development of novel therapeutics with improved specificity and reduced side effects.

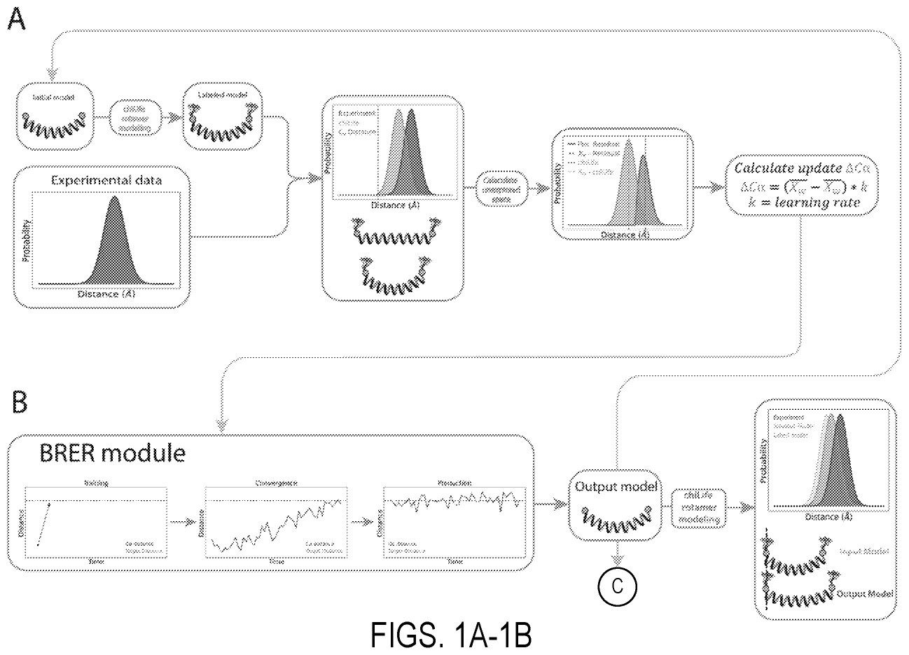

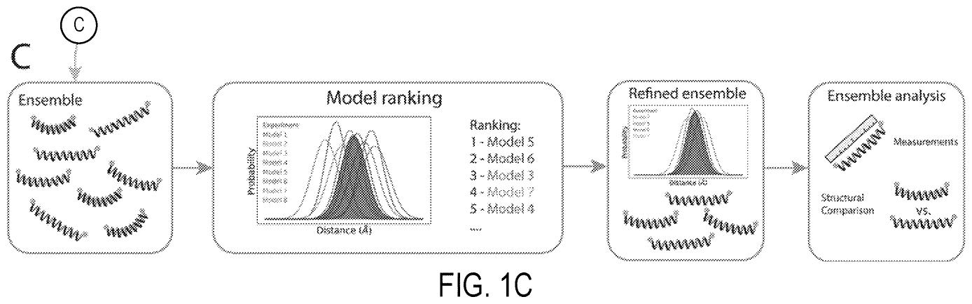

The framework presented here enables modeling of large global conformational rearrangements and residue-level microswitch rearrangements using multiple experimental distance distributions while minimizing overfitting errors. By way of non-limiting example, to achieve this, a three-block approach may be taken: an update block, a BRER block, and an analysis block, as illustrated in the workflow diagram in FIGS. 1A-1C.

FIG. 1A illustrates an example update block. This block provides an update to the Cα distance between the two spin-labeled residues. First, an input model and experimental data are fed into the framework. Using chiLife or another suitable in silico spin labeling technique, the simulated distribution between the two spin-labeled residues is calculated from the coordinates of the input model (orange distribution). The simulated distribution is compared with the experimental data and the unexplored space of the experimental distribution is calculated by subtracting the simulated distribution from the experimental data and keeping only the positive residuals (green). An update to the Cα distance is then calculated (as described below) and applied to the next block in the framework.

FIG. 1B illustrates a BRER block. This block performs the BRER methodology by loading the updated Cα distance and performing biased MD in 3 phases: training phase calculates a linear bias potential to drive the necessary change; convergence phase applies the bias potential to the system and ends once the distance between Cα atoms in the simulation is within a certain cut off (typically 1-2 Å) of the target distance; production phase allows the system to relax around this bias potential for some amount of time. Using chiLife, the last frame of the production phase is then used to simulate the spin-label distribution that this model provides. An example of the simulated distribution from the output model compared to the input model and experimental data is shown. The output model is then fed back in to the update block to calculate a new update to the Cα distance, and multiple frames are fed into the final block.

FIG. 1C illustrates a model ranking and analysis block. The ensemble of output models are re-labeled multiple times to generate spin label rotamer set/distribution that best overlays with the experimental data. The models are ranked (or can be reweighted) by the similarity of their simulated distance distribution to the experimental data. The selection criteria for the top n models then creates a modelled ensemble of the experimental data in the form of 3D protein models, which can be subject to further structural and statistical analysis. In some implementations, multiple separate ensemble runs may be performed to explore the conformational space defined by the experimental data. For example, four or more separate ensembles with 30 iterations each may be run, where each ensemble produces unique structural models that converge toward the target experimental distribution. The iterative modeling process progressively explores the conformational space, and as the number of iterations increases, the combined model distributions show increasingly better agreement with the experimental distance distributions. This approach may help ensure comprehensive sampling of the conformational landscape represented by the experimental data, as different ensemble runs may explore different regions of conformational space before converging on structures consistent with the experimental constraints.

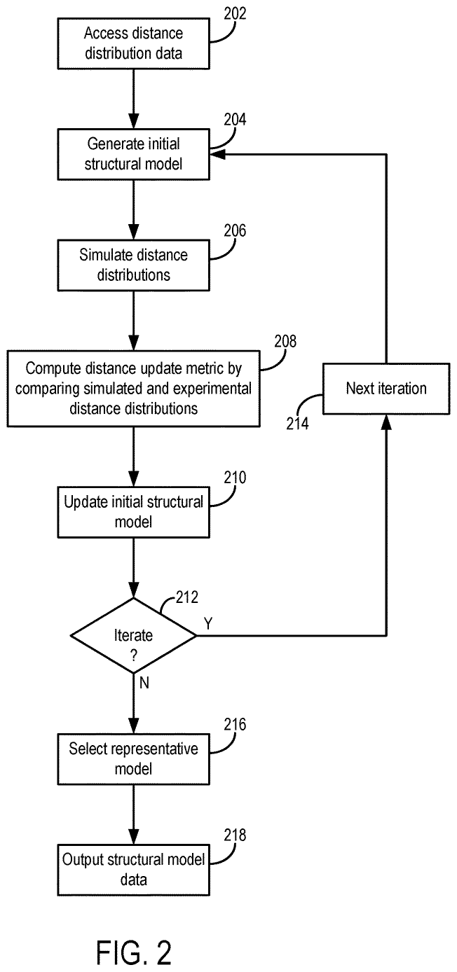

Referring now to FIG. 2, a flowchart is illustrated as setting forth the steps of an example method for modeling protein conformational ensembles using distance constraints obtained from spectroscopic measurements (e.g., CW EPR spectroscopy, DEER spectroscopy), which are used to measure distances between specific points in protein structures. The method may be used to derive distinct structural models of GPCRs, or other macromolecular signaling assemblies, stabilized by balanced and biased ligands. This approach may provide mechanistic insight into the pathway-specific efficacy of different ligands.

Advantageously, the method is capable of modeling protein conformational ensembles and their importance in determining protein functionality. In particular, the method is capable of generating accurate 3D protein models from spectroscopic data. The method includes a step-wise modeling approach to address large conformational changes and to accurately represent spin labels.

As described below in more detail, the method generally includes starting with an initial protein model (e.g., from cryoEM structures, crystal structures, AlphaFold predictions) and simulating the initial distance distribution (e.g., using in silico spin labeling techniques, such as those provided by chiLife). The simulated distance distribution is then compared with a distance distribution generated from experimental spectroscopy measurements (e.g., CW EPR data, DEER data). Updated Cα distances are calculated and biased towards in a biased resampling ensemble refinement (BRER) technique that is used to generate updated protein models.

The described computational modeling framework may allow for the modeling of large-scale global conformational rearrangements in proteins. By iteratively refining the protein models based on experimental distance constraints, the framework may generate structural ensembles that represent the conformational heterogeneity of the protein under different conditions or in the presence of different ligands.

By integrating these elements into a cohesive workflow, the method may create new opportunities for applying CW EPR and/or DEER spectroscopy techniques in drug discovery efforts. The approach may allow for screening of ligands and drug molecules based on protein conformational efficacy, potentially circumventing human biases in structure-based drug campaigns. This integrative pipeline can efficiently target highly dynamic proteins such as GPCRs, as well as drug pathological protein complexes and macromolecular signaling assemblies. The method may enable visualization of new protein conformations at atomic-level resolution, potentially enhancing structure-based drug discovery efforts by revealing conformations not observed using existing structural biology methods.

The method includes accessing one or more distance distributions with the computer system, as indicated at step 202. As used herein, the term “distance distribution” may refer to a probability distribution representing the range of distances between two specific points in a protein structure. The distances may be measured using spectroscopic techniques, including CW EPR spectroscopy, DEER spectroscopy, fluorescence resonance energy transfer (FRET), or the like. The distance distribution may reflect the conformational heterogeneity of a protein, showing the likelihood of observing particular distances between labeled sites. In some aspects, the distance distribution may be represented as a plot or curve showing the probability of different distance values, which may be measured in angstroms (Å). The shape, width, and peak positions of the distance distribution may provide information about the flexibility, conformational states, and structural changes of the protein under various conditions.

Accessing a distance distribution with the computer system may include retrieving a previously generated distance distribution from a memory or other suitable data storage device or medium. Additionally or alternatively, accessing a distance distribution may include generating the distance distribution with the computer system and then storing the distance distribution for later use by the computer system.

By way of example, a distance distribution may be generated by designing constructs of target proteins for spectroscopic analysis, collecting spectroscopic data from the constructs, and then analyzing the spectroscopic data to generate distance distributions, as described below in more detail.

As a non-limiting example, a protein construct in which a distance distribution may be collected via spectroscopic experiments (e.g., CW EPR, DEER) may first be designed by selecting appropriate sites for spin labeling. In some cases, GPCR constructs may be designed for CW EPR and/or DEER experiments. The design process may involve selecting specific amino acid residues for site-directed spin labeling. These residues may be chosen based on their location in regions of interest within the GPCR structure, such as transmembrane helices or intracellular loops. In some cases, the GPCR constructs may include mutations to remove native cysteine residues and introduce new cysteine residues at desired positions for spin labeling. These mutations may be introduced using site-directed mutagenesis techniques. The introduced cysteine residues may then be labeled with spin labels such as IAP, MTSL (1-oxyl-2,2,5,5-tetramethylpyrroline-3-methyl methanethiosulfonate), or the like.

The GPCR proteins may be expressed using mammalian expression systems. In some implementations, HEK293 cells may be used for protein expression. The cells may be transfected with plasmid DNA encoding the GPCR construct. Following expression, the proteins may be extracted from cell membranes using detergents.

Protein purification may be carried out using affinity chromatography. In some cases, the GPCR construct may include an affinity tag, such as a polyhistidine or FLAG tag, to facilitate purification. The purified proteins may then be reconstituted into membrane-mimicking environments. This reconstitution step may involve the use of detergents or lipid bilayers to maintain the native structure and function of the GPCRs.

A panel of ligands with varied pharmacological efficacy profiles may be used to study the conformational changes of the GPCRs. These ligands may include agonists, antagonists, inverse agonists, and biased agonists. In some implementations, FDA-approved drugs targeting the specific GPCR may be included in the ligand panel. For example, when studying the β2 adrenergic receptor, ligands such as isoproterenol (a full agonist), salbutamol (a partial agonist), propranolol (an antagonist), and ICI 118,551 (an inverse agonist) may be used.

The purified and reconstituted GPCR proteins may be incubated with these ligands prior to spectroscopic measurements. This incubation step may allow the GPCRs to adopt ligand-specific conformations, which can then be probed using DEER spectroscopy or other spectroscopic techniques. The spin-labeled GPCR samples may be prepared in buffer conditions optimized for DEER measurements. These conditions may include the presence of cryoprotectants such as glycerol to prevent ice crystal formation during freezing of the samples for DEER experiments.

With the protein constructs designed and prepared, spectroscopic data are then collected and analyzed to generate one or more distance distributions. The process of collecting DEER data, or other spectroscopic data, on ligand-bound receptors and generating distance distributions may involve several steps. In some cases, receptor preparations bound to various ligands with different pharmacological efficacy profiles are subjected to DEER experiments. The DEER data collected from these experiments may be analyzed to generate distance distributions between specific sites on the receptor.

To identify significant independent components in the DEER distance distributions, principal component analysis (PCA) and non-negative matrix factorization (NNMF) techniques may be employed. In some cases, PCA may be used to reduce the dimensionality of the DEER data and identify the main sources of variation in the distance distributions. NNMF may be applied to decompose the distance distributions into non-negative components, which may represent distinct conformational states of the receptor.

The combination of PCA and NNMF may allow for the determination of the number of significant independent components represented in the DEER distance distributions. In some cases, this factor analysis provides coupled distances and determines the number of independent conformations present in the receptor population. The distance constraints for each identified component may correspond to a distinct conformational state of the receptor.

By applying these analytical techniques to the DEER data, it may be possible to extract information about the conformational heterogeneity of the receptor in response to different ligands. This approach may provide insights into the structural basis of ligand-mediated signaling through native receptors.

Referring still to FIG. 2, an initial structural model of a macromolecular signaling assembly is constructed, as indicated at step 204. As used herein, the term “macromolecular signaling assembly” may refer to a large complex of multiple proteins and/or other biomolecules that work together to transmit cellular signals. These assemblies may include receptors, enzymes, scaffolding proteins, and other components that interact to propagate and regulate signaling pathways within and between cells. In some aspects, macromolecular signaling assemblies may be dynamic structures that can change their composition or conformation in response to stimuli. The term may encompass complexes involved in various cellular processes such as signal transduction, metabolic and immune regulation, or gene expression. As one non-limiting example, a macromolecular signaling assembly may include a GPCR or GPCR signaling complexes, such as those involving the β2 adrenergic receptor (β2AR) or the angiotensin II type 1 receptor (AT1R). These may include the receptor itself, associated G-proteins, ligands, and other regulatory molecules that modulate receptor function and downstream signaling. As another example, a macromolecular signaling assembly may include GPCR-β-arrestin complexes, which may form upon receptor activation and are involved in signal termination and alternative signaling pathways. As yet another example, a macromolecular signaling assembly may include ligand-receptor-effector protein assemblies, such as those formed when different ligands (e.g., balanced agonists, biased agonists, or inverse agonists) bind to GPCRs or other receptors, leading to distinct conformational states and signaling outcomes. As still another example, a macromolecular signaling assembly may include protein complexes involved in GPCR-mediated, or other, signal transduction pathways, which may include multiple interacting proteins that transmit and amplify signals from the receptor to various cellular effectors.

The initial structural model may include a three-dimensional representation of the atomic structure or backbone conformation of a protein or other macromolecular signaling assembly. These models may be derived from various sources such as x-ray crystallography, cryo-electron microscopy, or nuclear magnetic resonance (NMR) spectroscopy. In such instances, generating the initial structural model may include accessing, with the computer system, x-ray crystallography data, cryo-electron microscopy data, NMR spectroscopy data, or other such data from which the initial structural model may be generated. Additionally or alternatively, the initial structural model(s) may be generated using computational protein structure prediction methods such as AlphaFold, Rosetta, or the like.

The initial structural model serves as a starting point for refinement using experimental distance constraints. Advantageously, the initial structural model may represent different conformational states of a protein or macromolecular signaling assembly, such as active, inactive, or intermediate forms. Structural models may be visualized as ribbon diagrams, atomic-level structures, or the like, and may be used to analyze protein function, predict ligand binding sites, and/or guide structure-based drug design efforts. In some cases, ensembles of structural models that collectively represent the conformational diversity of a protein as informed by experimental distance measurements may be generated, as described below in more detail.

One or more simulated distance distributions are then generated using the initial structural model, as indicated at step 206. In some cases, chiLife or another suitable in silico spin labeling package may be used to estimate spin-spin distances from a given protein model. Additionally or alternatively, chiLife or another suitable in silico spin labeling package could be used to build and model probes that are used in other spectroscopies to measure distances, such as FRET. The chiLife package may simulate the attachment of spin labels to specific residues in the protein structure contained in the initial structural model and generate a distribution of possible spin label conformations. By way of example, chiLife may be used to model spin label side-chain rotamers on the protein and create a simulated distance distribution for each spin-pair used in the spectroscopic experiments. From these simulated spin label conformations, estimated distances between spin label pairs may be calculated.

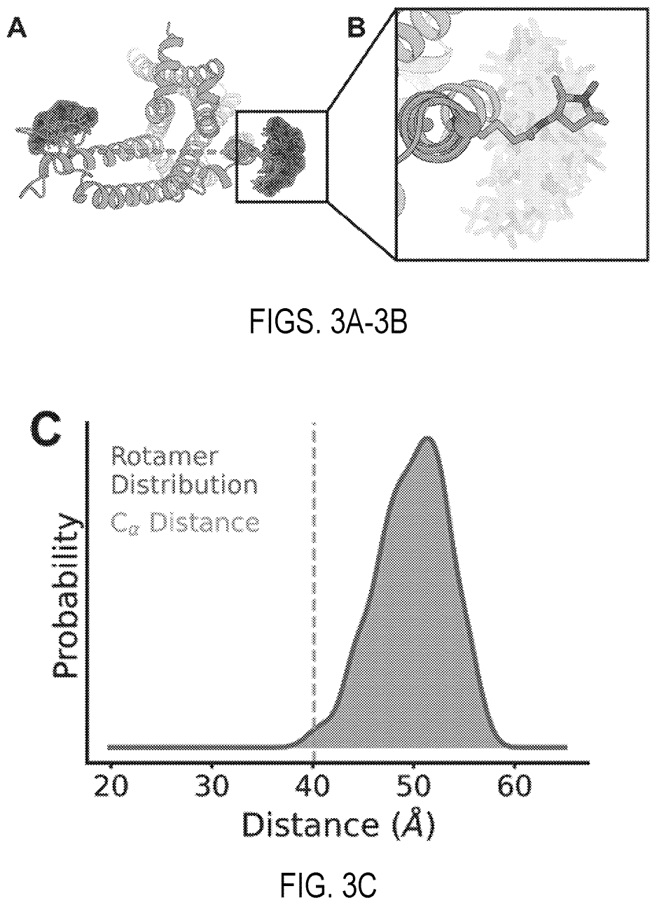

FIGS. 3A-3C illustrate an example of spin-label rotameric heterogeneity. FIG. 3A illustrates IAP rotamers (green) modeled on to the β2AR (grey cartoon, PDB ID: 3SN6, loops modelled in), shown from the cytoplasmic face. Transparent clouds represent the nitroxide N—O atoms where the free radical is trapped, which ultimately serves as the point of measurement in DEER spectroscopy. Orange spheres represent the C_{α} atoms of spin labeled residues, with an orange dashed line signifying the distance between C_{α} atoms. FIG. 3B shows a close up on the IAP spin label, with a single rotamer highlighted. FIG. 3C is a graph of the IAP nitroxide distribution (green) and distance between C_{α} atoms of the IAP-labeled residues (orange dotted line).

The one or more simulated distance distributions are then compared with the one or more experimental distance distributions to generate a distance update metric, as indicated at step 208. The distance update metric may represent the difference between the estimated distances and experimental constraints.

By way of example, a simulated distance distribution may be subtracted from the experimental distribution and a positive residual function (PRes) may be applied to the resulting residual distribution vector. In some cases, the PRes may be implemented as a Rectified Linear Unit (ReLU) function. As a non-limiting example, the PRes function may be calculated according to:

$$PREes ( {P}_{exp} , {P}_{model} ) = \frac{( {P}_{exp} - {P}_{model} ) + \vert {P}_{exp} - {P}_{model} \vert}{2} ; \qquad ( 1 )$$

where Pₑₓₚ is the probability vector of the experimental probability-distance distribution and P_{model} is the probability vector of the simulated probability-distance distribution from the input model protein. The PRes function returns all positive values and replaces negative values with zero, thus identifying all areas of the experimental distribution that are not sampled by the input model distribution. This may be referred to as the positive residual distribution. A momentum parameter (ρ) may also be specified (defaults to 1) to control how many previous models to incorporate into the positive residual distribution calculation. When incorporating multiple previous models, the PRes function may be calculated as:

$$PREes ( {P}_{exp} , {P}_{model} , ρ ) = \frac{( {P}_{exp} - \frac{1}{ρ} {\sum }_{1}^{ρ} {P}_{model - ρ} + \vert {P}_{exp} - \frac{1}{ρ} {\sum }_{1}^{ρ} {P}_{model - ρ} \vert )}{2} ; \qquad ( 2 )$$

where ρ indicates the momentum parameter, P_{model-1} is the probability vector from the simulated probability-distance distribution of the latest generated model, P_{model-2} is the model generated prior to P_{model-1}, and so on. The

$$\frac{1}{ρ}$$

term is a normalization term, assuming Pₑₓₚ and P_{model} are already area normalized to one another. A value greater than 1 for the momentum parameter is useful in cases wherein the width of the input model distribution is narrower than the experimental distribution so that the entirety of the experimental distribution is explored.

The probability weighted average distance of a probability distance distribution may be calculated as follows:

$$X ( r , P ) = \frac{{\sum }_{i} {r}_{i} {P}_{i}}{{\sum }_{i} {P}_{i}} . \qquad ( 3 )$$

To determine the distance constraint used in BRER simulations, an update to the Cα distance (ΔCα) may be calculated according to:

ΔCα=k(X̅_̅{̅w̅,̅r̅e̅s̅}̅−X̅_̅{̅w̅,̅m̅o̅d̅e̅l̅}̅) (4);
where X̅_̅{̅w̅,̅r̅e̅s̅}̅ and X̅_̅{̅w̅,̅m̅o̅d̅e̅l̅}̅ are probability-weighted average distances of the positive residual and input model distributions, respectively, and the update rate k is a value between 0 and 1 used to control the size of the update applied. In this application, a value lower than 1 reduces the magnitude of the change in distance between Cα atoms imposed in BRER simulations, providing for relatively small perturbations to the model. The calculated ΔCα is then added to the distance between Cα atoms of spin-labeled sites in the input model to produce the target distance, specified as:

Cα_{target}=Cα_{model}+ΔCα (5).

The target distance Cα_{target} and input model are then fed into the BRER biased MD engine.

The initial structural model may then be updated using the distance update metric, as indicated at step 210. As an example, the calculated distance update metric may be used as a distance constraint in BRER simulations. For instance, ΔCα may be added to the distance between Cα atoms of spin-labeled sites in the input model to produce the target distance, specified as:

Cα_{target}=Cα_{model}+ΔCα (6).

In some cases, applying the distance update metric to the initial structural model may be performed using a linear bias potential in a molecular dynamics simulation. The linear bias potential may drive the Cα atoms of the spin labeled residues towards the target distances determined by the update metric. This approach may allow for incremental changes in the backbone configuration of the protein while maintaining overall structural integrity. The result of this simulation may be a new protein model that more closely aligns with the experimental data.

BRER follows three phases to bias the input model to experimentally derived constraints: training, convergence, and production phases. The training phase determines a linear bias potential needed to drive the Cα-Cα distances to the targets determined in the Cα update step. In the case of multiple experimental constraints, linear bias potentials for all constraints may be optimized simultaneously during the training phase. Once training is completed for a particular constraint, the bias potential is extracted and training continues for the remaining constraints. The convergence phase applies the learned bias potentials to the system until Cα distances converge onto the target distances, at which point the system is taken to the production phase. In the production phase, the simulation continues for a user-specified amount of time with bias potentials intact, which allows the system to relax to the abrupt structural transition imposed during convergence. The last frame in the production phase is used as an output model (e.g., as illustrated in FIG. 1B).

The newly generated protein model may then be used as the input for a subsequent round of distance estimation and update calculations, as indicated by the iterative loop with decision block 212 where a subsequent iteration of the model refinement may be initiated at process block 214. This process may create an iterative approach that produces incremental changes in the backbone configuration of the structural model. Each iteration of the update and BRER blocks (e.g., as illustrated in FIGS. 1A and 1B) use the output model of the prior iteration as an input model.

In some cases, the structural models generated at each iteration may become part of a collective ensemble. This ensemble may explore the conformational space represented by the input experimental data. Multiple rounds of chiLife rotamer modeling may be performed on each structural model in the ensemble to determine the rotamer set that best fits the experimental data.

For example, the process described above may be repeated n times to generate an ensemble of models (e.g., as illustrated in FIG. 1C) that includes a number of frames (e.g., 10 frames) from the last ns of the production phase (one every 0.1 ns) from each iteration. The resulting ensemble explores the entire conformational space represented by the experimental data, even when the starting model differs significantly from the conformation captured in experiments. Additionally, the majority of models exhibit minimal changes of secondary structure in regions local to sites where the bias potential is applied, where these changes would represent an over fitting of the data.

A representative model for the macromolecular signaling assembly may be selected from the ensemble of models, as indicated at step 216. The models in the ensemble may be ranked according to their degree of overlap with the experimental data. In some cases, this ranking may involve calculating a similarity metric between the simulated distance distributions and the experimental distance distributions for each model. For example, to select representative models that agree with the experimental data, each model distribution is ranked against the experimental data by means of the Jensen-Shannon (JS) distance or inverse dot product. The JS distance is calculated as:

$$\sqrt{\frac{D ( p ‖ m ) + D ( q ‖ m )}{2}} ; \qquad ( 7 )$$

where p and q are the modeled and experimental distributions, D is the Kullback-Leibler divergence, and m is the point-wise mean of distributions p and q. Alternatively, similarity between modeled and experimental distributions can be calculated via the inverse dot product:

$$\frac{1}{p \cdot q} ; \qquad ( 8 )$$

where the magnitude of the dot product is proportional to the similarity of the two vectors. A smaller value for JS distance or inverse dot product indicates greater similarity between model and experimental distributions.

The top n ranked models may be summed together to create a final ensemble that is compared to the experimental distributions by the JS distance. In an example study, for the ATIR, ranking models using the inverse dot product was found to produce a smaller JS distance in the top 50 summed models compared to the experimental data. In the example study, for the β2AR, ranking models using the JS distance resulted in a final ensemble that most agreed with the experimental data.

The generated structural models may be output using the computer system, as indicated at step 218. For example, the generated structural models may be analyzed and visualized using various methods to extract meaningful information about protein conformations and ligand-induced effects and the results of these analyses may be output to a user by the computer system

In some cases, structural analyses are conducted on the top-ranked models to identify conformational changes and key structural features. These analyses may include measuring distances between specific residues, calculating root-mean-square deviation (RMSD) values between different models or with reference structures, and examining changes in secondary structure elements. In some implementations, functionally relevant structural motifs may be analyzed across the dataset to evaluate the statistical significance of conformational differences between components.

Principal component analysis (PCA) may be used to demonstrate that global conformational rearrangements are non-random and distinguishable between datasets. PCA may be performed on the coordinates of protein backbone atoms or specific subsets of atoms, such as those involved in ligand binding or signaling. The resulting principal components may reveal the major modes of conformational variation within the ensemble of models. In some cases, projecting the models onto the principal component space allows for visualization of distinct clusters corresponding to different conformational states or ligand-induced effects.

To aid in interpretation and comparison of different conformational states, representative models from each component or cluster may be selected for detailed visualization, as described above. These models may be overlaid to highlight structural differences and rearrangements in specific regions of interest. In some implementations, multiple representative models may be used to create ensemble representations that capture the conformational heterogeneity within each component.

Statistical analyses may be performed on larger sets of representative models to quantify the significance and magnitude of observed conformational changes. These analyses may include calculating mean distances, angles, or other structural parameters, as well as their associated standard deviations or confidence intervals. In some cases, statistical tests may be used to determine whether differences between components or ligand-induced states are significant.

The results of these analyses and visualizations may provide insights into the conformational efficacy of different ligands, the structural basis of biased signaling, and the dynamic behavior of the protein in response to various stimuli. This information may be valuable for understanding protein function, identifying potential drug binding sites, and guiding structure-based drug design efforts.

In an example study, the systems and methods for modeling protein conformational ensembles using spectroscopic distance constraints described in the present disclosure were used to generate structural models of the angiotensin 1 type II receptor and the β2 adrenergic receptor. As described above, the disclosed methods implement BRER and chiLife for the use of multiple experimental distance distributions to drive large and complex global conformational rearrangements and residue-level microswitch rearrangements.

BRER is an iterative ensemble refinement method that utilizes biased MD simulations implemented in GROMACS 2019.6 using gmxapi. Briefly, BRER creates new models by means of a three-phase process: training, convergence, and production. A learned maximum-entropy linear bias potential (training phase) is applied to the whole system and drives two atoms to a target distance (convergence phase), after which the applied linear bias is maintained for a user-specified period of time to allow the rest of the system to relax (production phase).

In the example study, the BRER framework was modified using a Python wrapper to eliminate the probability weighted draw from the input distribution and instead utilize a single target distance between each atom pair. Identification of target distances is described above. The system inputs used here are a starting MD system and target distances.

Primary adjustable parameters include A, tolerance, and τ (tau). A is the learning rate for the coupling constant α (i.e., bias potential), and defines the initial search space of the coupling constant α. The A value is a critical parameter for determining convergence and behavior of the system. A values set too small will not enable proper convergence on the target distance, and values set too large may produce erroneous models and system parameters due to a highly dissipative convergence process. The ideal value of A is the maximum value of α expected for the given transformation. If a larger coupling constant is needed, the program will search above the set A value in the training phase until a suitable α is found. Tolerance specifies how close the distance between atoms has to be to the specified target distance, in nm, before the simulation moves from the convergence to the production phase. The τ value describes the length of time in ps the system is allowed to adapt to the new a in the training phase before either re-adjusting the α value or accepting the current value and moving to the convergence phase. The τ value can have an impact on the learned α, where higher values of τ introduce lower coupling constant values. The parameters of A, tolerance, and τ used in this example study are provided in Table 1.

TABLE 1
Parameters used in the modeling pipeline

| | A | τ (ps) | Tolerance (nm) | Production time (ns) |
|---|---|---|---|---|
| β2AR modelling | 100 | 1000 | 0.1 | 5000 |
| AT1R modelling | 200 | 1000 | 0.1 | 10000 |

ChiLife is an opensource Python package used to simulate distance distributions of EPR spin labels attached to protein structures/models in-silico, and is highly amenable to unique spin labels, rotamer libraries, and rotamer sampling methods. For the IAP label, used herein on the β2AR, the rotamer library was expanded through a modified version of off-rotamer sampling in which spin label side chain dihedral angles were randomly altered using a standard deviation of 15° from the underlying rotamer library. For the V1 label used on the ATIR, the rotamer library was expanded in the same manner with dihedral angle standard deviation of 10°. The dihedral angle standard deviation values specified above were the minimal values that generated reproducible in-silico labeling and distance distributions. The rotamer sets created using this approach were used for their respective proteins through the entirety of model generation and analysis.

In all cases, CHARMM-GUI was used to set up the initial system, with missing loop regions modelled in using Modeller in ChimeraX. For the β2AR, the carazolol- and inactive-state stabilizing nanobody NB60-bound crystal structure was used as the starting model (PDB ID 5JQH). The N terminal T4 lysozyme addition, NB60 nanobody, and carazolol were removed. Mutations consistent with the β2AR construct used in the DEER experiments were introduced: C77V, C265A, C327S, C341L, C378A, C406A, M96T, and M98T. Titratable residue protonation assignments were determined at pH 7, with exception of the following residues which were protonated consistent with previous studies: E107, E122, D130, H172, E268. Disulfide bonds were specified between residues C184/C190 and C106/C191. Box dimensions were set to 90 Å×90 Å with 22.5 Å above and below the bilayer composed of 201 POPC lipids and solvated with 20,358 water molecules. 0.15 M NaCl was added in neutralizing conditions for a total of 55 Na and 66 Cl ions.

For the ATIR, the 4YAY crystal structure was used as the starting model with the extracellular BRIL truncated. The conditions used to set up this system were consistent with those present in Wingler L M, et al., “Angiotensin Analogs with Divergent Bias Stabilize Distinct Receptor Conformations,” Cell. 2019; 176 (3): 468-478 (“Wingler 2019”). Hydrogens were added to the structure and titratable residues were protonated in accordance with their dominant protonation state at pH 7. Residues D74 and D125 were deprotonated, consistent with observations in other GPCRs. N and C termini capping was added with acetyl (ACE) and methylamide (NT3) groups, respectively. Disulfide bonds were specified between residues C101/C180 and C18/C274. An 80×80 Å box was created composed of 153 POPC lipids and solvated with 85 water molecules per lipid for a total of 13,005 water molecules. 0.15 M NaCl was added in neutralizing conditions for a total of 41 Na and 55 Cl ions.

GROMACS was used for all MD simulations and CHARMM36m was used as the forcefield in all simulations with a TIP3P water model. Relaxation using steepest descent was performed to a tolerance of 1.195 kcal/mol/Å². Positional restraints were set at 10 kcal/mol/Å² and an initial equilibration was performed for 20 ns using a 1 fs timestep. For the β2AR and ATIR, restraints were decreased to 5 kcal/mol/Å² and tapered off across 60 ns and 80 ns, respectively, using NPT conditions with a velocity rescaling thermostat and a C-rescale barostat and a leapfrog integrator with a 2 fs timestep. Restraints were removed and each system was allowed to equilibrate for 50 ns in which the starting model for BRER was selected. All hydrogen atom bond lengths were constrained using the LINCS algorithm. All short-range electrostatics and Van der Waals interactions were calculated with a 12 Å cutoff and switching at 10 Å. All long-range electrostatic interactions were computed with the Particle-Mesh-Ewald method.

After model creation, spin label side chain rotamers were simulated five times for each spin pair and the resulting modeled distance distribution with the lowest JS distance from the experimental distribution was chosen for that model and spin pair. This approach was used to account for variability in the chiLife repacking method, which can introduce heterogeneity in modeled distributions due to the Markov Chain Monte Carlo (MCMC) process of adding label rotamers. A 3D array was created in the shape models x spin pairs x distance distributions for each NMF component modelling run. This was reshaped to yield a 2D array of dimensions models x spin pairs*distance distributions, such that each distribution in a single model is a part of the same vector. Each model in the 2D array is then ranked by the JS distance or inverse dot product from the experimental data reshaped into a 1D array of connected distance distributions, where the top n models are summed to represent the experimental data.

Within the top n models, secondary structure local to the label sites are analyzed qualitatively for alterations that indicate overfitting. In the presence of overfitting, model selection criterion is adapted to include a filter for excluding the change in secondary structure. For the β2AR, approximately 600 of the models were over fit by showing a helix-to-loop transition at the cytoplasmic surface of TM6 at the site of the applied bias. These models were filtered out using HELANAL in MDAnalysis to calculate a local helix height sum of <11 Å from residues 267-275, leaving approximately 1200 models with the cytoplasmic end of TM6 in an alpha helix. No filter was used for the ATIR models. Following the filtering, the models are re-ranked according to the JS distance or inverse dot product as mentioned in the previous paragraph.

DEER data for β2AR 148/266 IAP was published in Lerch M T, et al., “Viewing Rare Conformations of the β2 Adrenergic Receptor with Pressure-Resolved DEER Spectroscopy,” Proc Natl Acad Sci USA, 2020; 117 (50): 31824-31831. In this publication, the active conformation of TM6 populated by epinephrine+NB80 is represented as a gaussian centered at 50 Å, which was used herein as input data for modeling. DEER data for the ATIR was published in Wingler 2019, included ten spin pairs and ten different liganded and unliganded conditions. Non-negative matrix factorization (NNMF) resolved four unique components (i.e., conformations) represented in the distance distribution data. Distance constraints from each of the four components were used here in modeling.

The ability of the disclosed methods to capture a large, complex conformational rearrangement using a single spin pair on the β2 adrenergic receptor (β2AR), which is a well-studied class A GPCR with available structures in various conformational states, was observed. Its structural rearrangements span up to 10 Å in non-loop regions, where maintaining secondary structure is crucial due to the prevalent allosteric communication between helices of GPCRs. Given its extensive characterization, the β2AR serves as an excellent test system for this method. We used previously published DEER data on β2AR 148/266 IAP bound to agonist isoproterenol and nanobody-80 (Nb80)—an active state stabilizing nanobody—as the target distribution beginning from an inactive β2AR conformation. This construct has been used in multiple DEER studies to report the conformational equilibrium of transmembrane helix (TM) 6, which is the helix exhibiting the largest conformational rearrangement between inactive and active states.

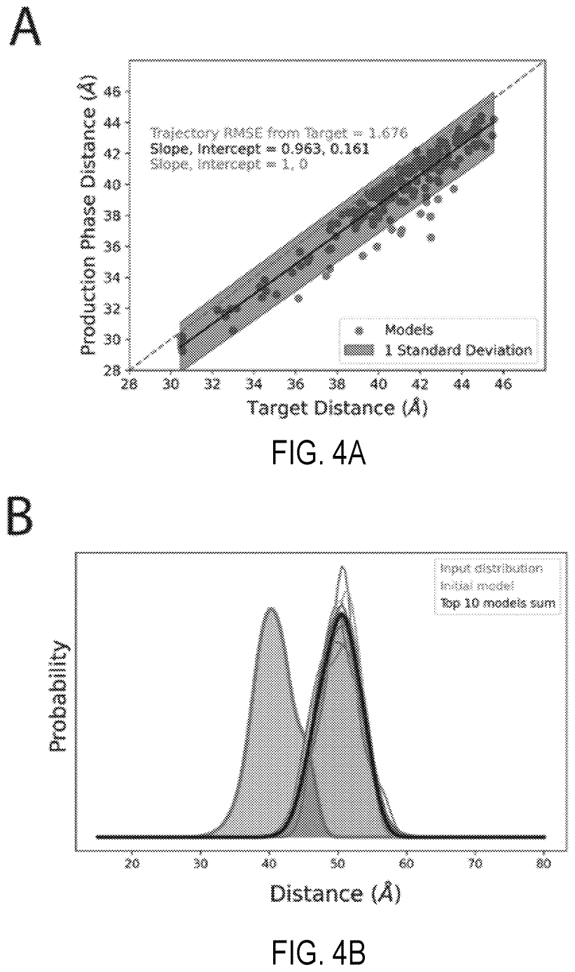

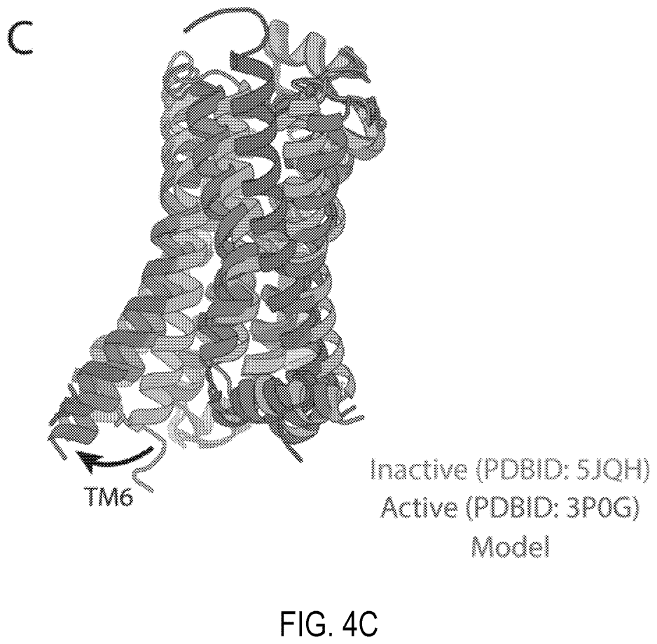

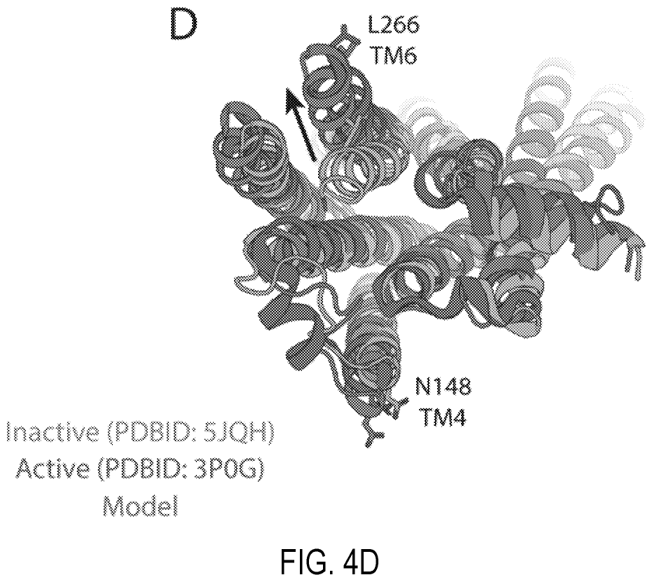

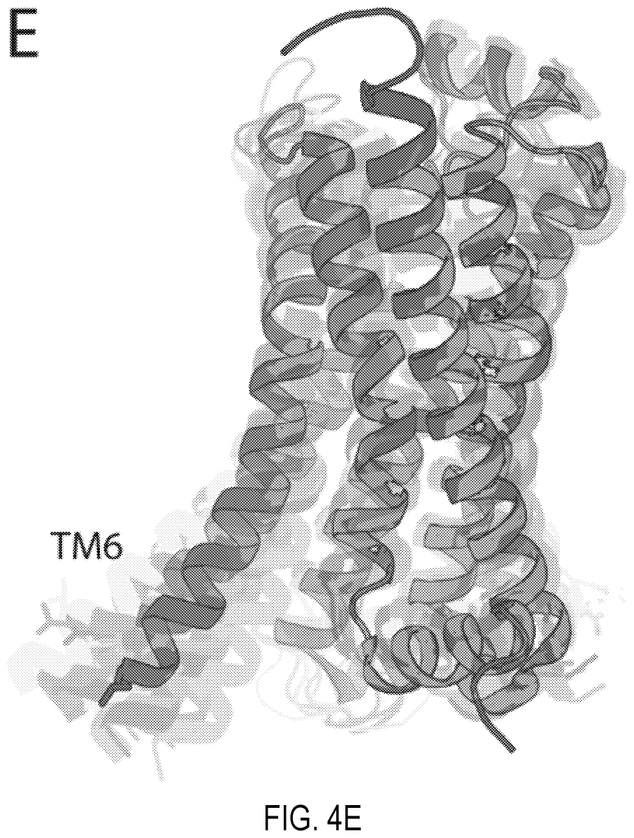

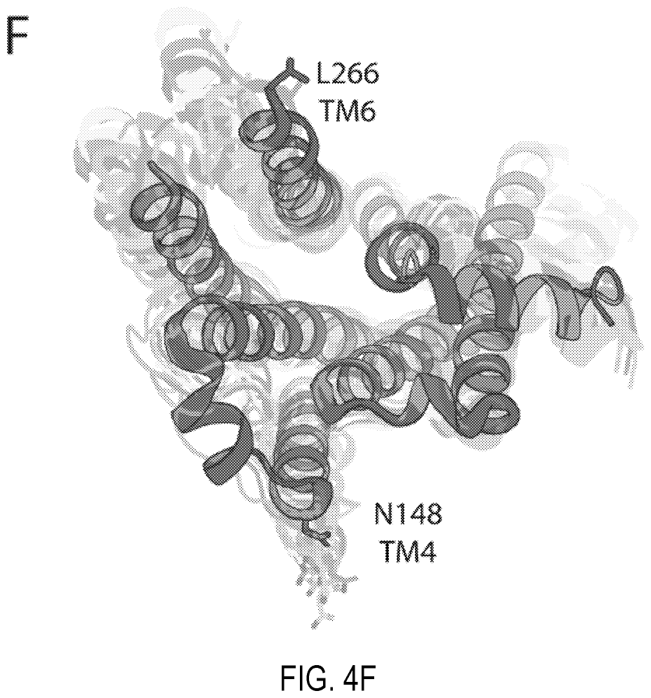

Model generation of the β2AR was performed in six individual runs with 30 iterations each (180 total) using an inactive structure as the starting model. Following training and convergence of the distance update in the BRER block, a production phase of 5 ns was used to allow relaxation around the bias constraints following the structural perturbation. To assess how well the BRER-generated models matched the distance constraints, we compared the target Cα-Cα distances with the average Cα-Cα distances during the production phase. The RMSE between these values is 1.676 Å, indicating good agreement between the generated models and the target distances. A plot of the average production phase Cα-Cα distance vs the target Cα-Cα distance for each of the 180 iterations is shown in FIG. 4A. The production phase distances are systematically lower than the target distances, likely due to the modeled distance change—an ~10 Å increase in the Cα-Cα distance from the starting model.

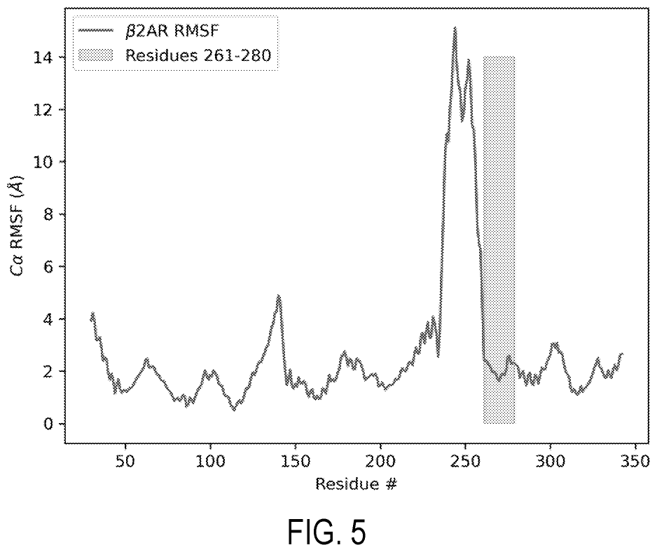

Ten frames from the last ns of the production phase (one every 0.1 ns) from each iteration were used as models in the analysis block, for a total of 1800. The models were ranked by JS distance and a final ensemble comprised of the top ten models was used for structural analysis. Simulated distance distributions for each of the top ten models demonstrate good agreement with the target distance distribution, and the sum of the top ten models achieves a JS distance of 0.04, indicating nearly perfect overlap with the experimental data (FIG. 4B). A representative model is overlayed with β2AR inactive state (i.e., starting model) and active state (i.e., target structure) in FIGS. 4C and 4D, highlighting the ~10 Å shift of TM6 from an inward inactive position to an outward active position while minimizing local secondary structure changes. Though all top 10 models show similar rearrangements of TM6, some models deviate further from the overlayed β2AR crystal structure demonstrating conformational heterogeneity that is still consistent with the experimental data (FIGS. 4E and 4F). To quantify this heterogeneity a Cα RMSF plot was created using the top 10 models (FIG. 5) where the residue specific Cα deviation from an average model is easily visible. In TM6 from residues 261-280, the mean Cα RMSF is 2.35 Å and the RMSF at site 266 is 2.13 Å.

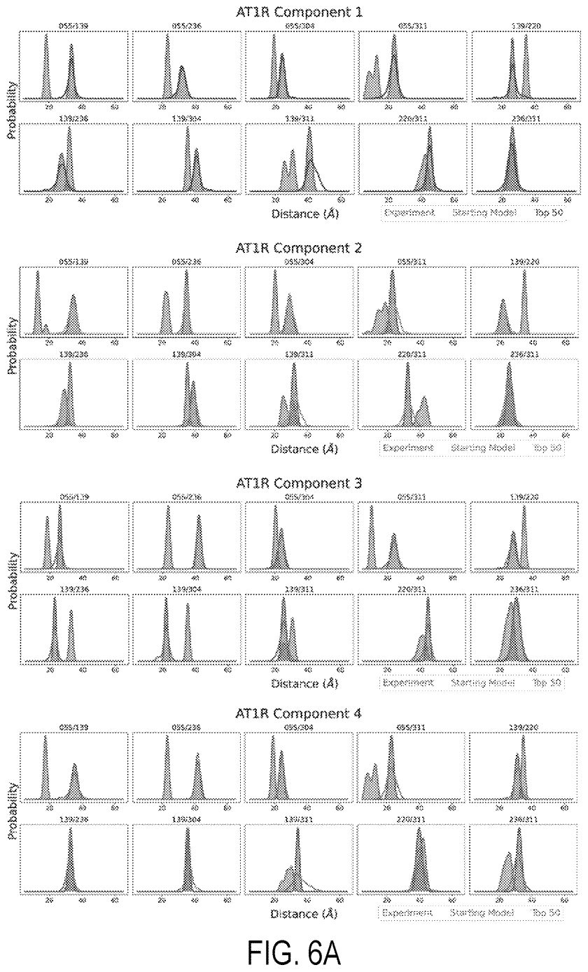

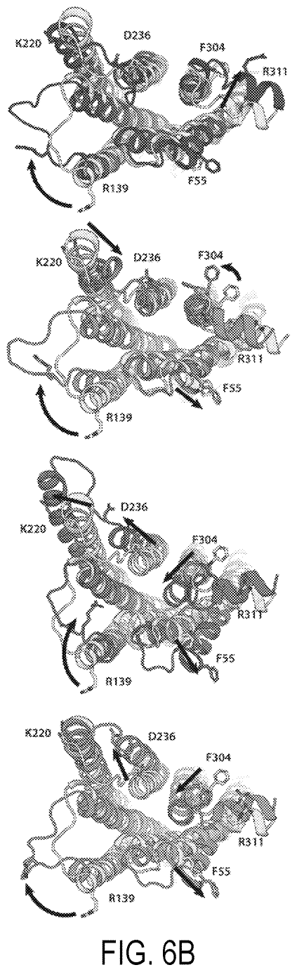

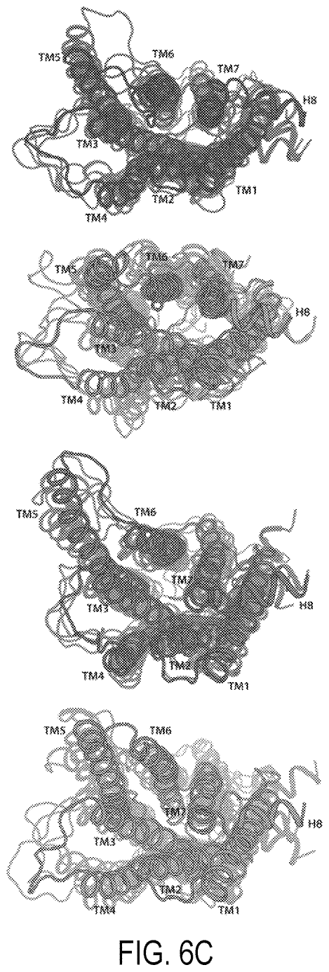

To showcase the capability of the disclosed framework to model conformational rearrangements using constraints from multiple spin pairs, it was applied to the ATIR utilizing DEER data reported by Wingler 2019. The DEER dataset included 10 spin pairs distributed across the cytoplasmic surface, which were used to monitor conformational changes induced by ligands with distinct efficacy profiles (i.e., inverse agonism, balanced agonism, β-arrestin biased agonism, or Gq biased agonism) to provide insight into the structural basis for biased agonism. This study identifies a set of four distinct conformations whose relative populations are modulated by ligands in correlation with their functional profiles. To visualize and compare the different conformations, spin density clouds were calculated and projected onto published structures. Here, distance constraints from the ATIR DEER data were used to generate complete structural models of each of the four conformations. Using an inactive state starting model, eight modelling runs with 30 iterations were performed for each component. Because of the potential of an individual run getting stuck in a single energetic well, the use of multiple replicates increases exploration of the conformational space available to the receptor. A comparison of the target distances with average production distance between labeled Cα residues in the ATIR conformations 1-4 showed RMSE values of 0.993 Å, 1.398 Å, 0.863 Å, and 1.379 Å, respectively. The top 50 models were summed together linearly and used to represent the final distributions for each component (FIG. 6A).

Excellent agreement is observed in the most probable distance for the modeled and experimental distributions, even in cases where this required a shift of ~20 Å from the starting model (e.g., 55/139-V1 in component 3). This highlights the ability of this framework to produce coordinated structural rearrangements driven by multiple distance constrains. Good agreement is also found in the widths of most modeled and experimental distributions, with the exception of the 139/311-V1 pair. Residues 139 and 311 are found in ICL2 and helix 8, respectively, which are highly dynamic regions of the receptor. A comparison of the target distances with average production distance between labeled Cα residues in the ATIR conformations 1-4 showed RMSE values of 0.993 Å, 1.398 Å, 0.863 Å, and 1.379 Å, respectively, indicating good agreement between the generated models and the target distances.

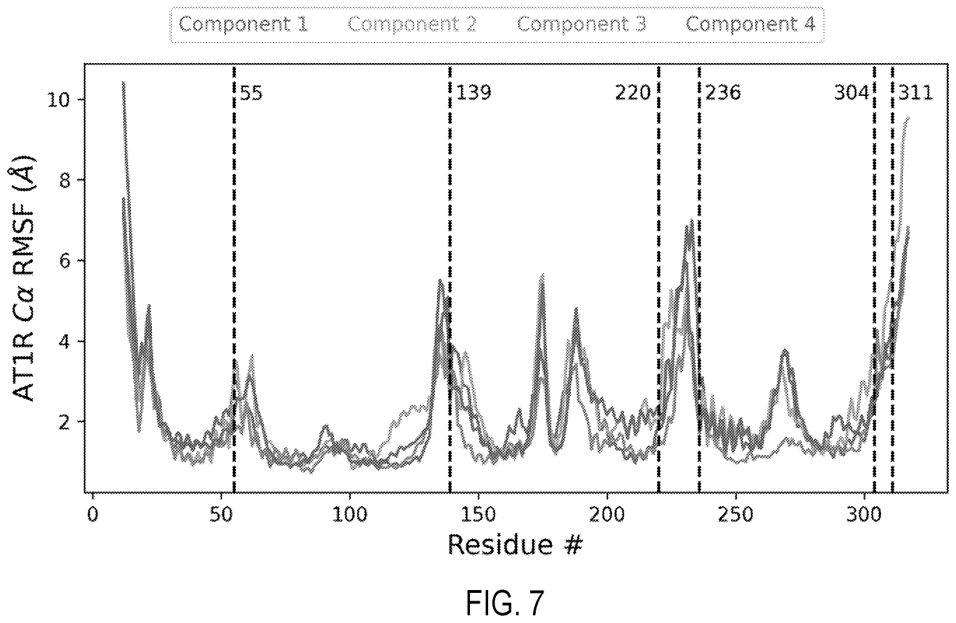

Overlays of the starting ATIR model with representative models for each conformation are shown in FIG. 6B to illustrate the structural rearrangements on the cytoplasmic surface. Major structural changes for each conformation are highlighted (black arrows, FIG. 4B) by the Cα atom of the spin site in the corresponding loop or helix moves by >3 Å. All distances referenced below indicate changes in spin site Cα position between the starting model and the aligned representative model conformation. The primary and most notable rearrangement is found in intracellular loop 2 (ICL2), driven by an increase in the distance for the 55/139-V1 spin pair. Conformations 1, 2, and 4 exhibit an outstretched ICL2 that is displaced from the cytoplasmic bundle, whereas ICL2 is positioned near the helical core in conformation 3. Conformation 1 shows a 3.2 Å outward shift in residue 304 (TM7) as the primary conformational rearrangement. Conformation 2 displays a 3.9 Å inward displacement of residue 220 (TM5) towards the center of the helical bundle, a 6.9 Å outward and upward shift of residue 236 (TM6), a 3.7 Å outward displacement of residue 304 (TM7), a 3.6 Å outward shift in residue 55 (TM1), and a 13.2 Å shift in residue 311 (H8). Conformation 2 showed a consistent displacement of residue 311 (H8) away from the plane of the membrane, as well as the only conformation to show an upward displacement in TM6. This represents a novel β-arrestin-biased conformation with unique structural features including vertical displacement of TM6 and H8 displaced from the plane of the membrane. Component 3 models also show a significant inward transition of residue 304 (TM7) by 7.1 Å, likely accommodated by the outward shift in TM1. The starting model 55/304 Cα-Cα distance lies within the range of the conformation 3 distance range for this spin pair (Figure S3), yet the overlayed models show a significant change in the two helices containing these residues due to the concomitant changes in other spin pairs involving residue 55 and 304. Conformation 4 shows a significant outward displacement of 6.9 Å in residue 236 (TM6) and 6.4 Å in residue 55 (TM1), whereas residue 304 (TM7) shows a 5.3 Å shift towards the helical core, again likely accommodated by the outward movement in TM1. To aid in quantifying the heterogeneity at these sites, the RMSF of each label site Cα for the top 50 models of each component are listed in Table 2 where the conformational rearrangements mentioned above are all greater than their respective RMSF values at each site (RMSF plot in FIG. 7).

TABLE 2
RMSF values of spin site Cα atoms for the top 50 models in each
component. Components are listed as row indices and spin sites are
listed as column indices. Values in the table are in angstroms (Å).

| | Site | | | | | |
|---|---|---|---|---|---|---|
| Component | 55 | 139 | 220 | 236 | 304 | 311 |
| 1 | 3.02 | 2.84 | 1.90 | 2.86 | 2.53 | 4.12 |
| 2 | 3.28 | 2.51 | 1.89 | 1.84 | 3.64 | 5.63 |
| 3 | 1.74 | 2.39 | 1.54 | 2.24 | 2.59 | 3.56 |
| 4 | 2.15 | 3.19 | 2.38 | 1.71 | 2.44 | 4.68 |

The top 5 models generated from each component (FIG. 6C) serve to show the heterogeneity that is present in the ensemble for each conformation.

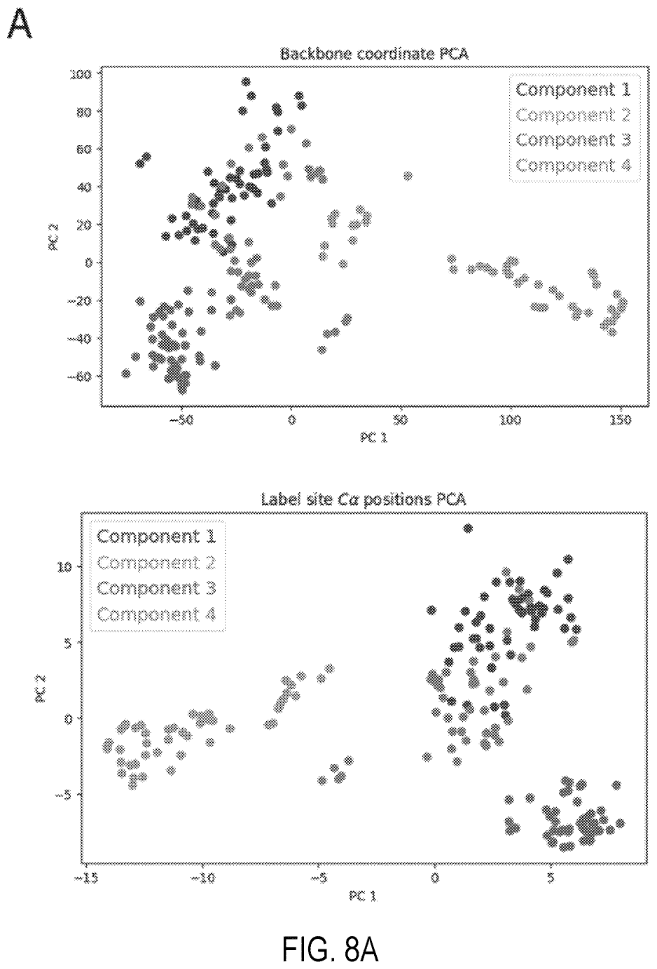

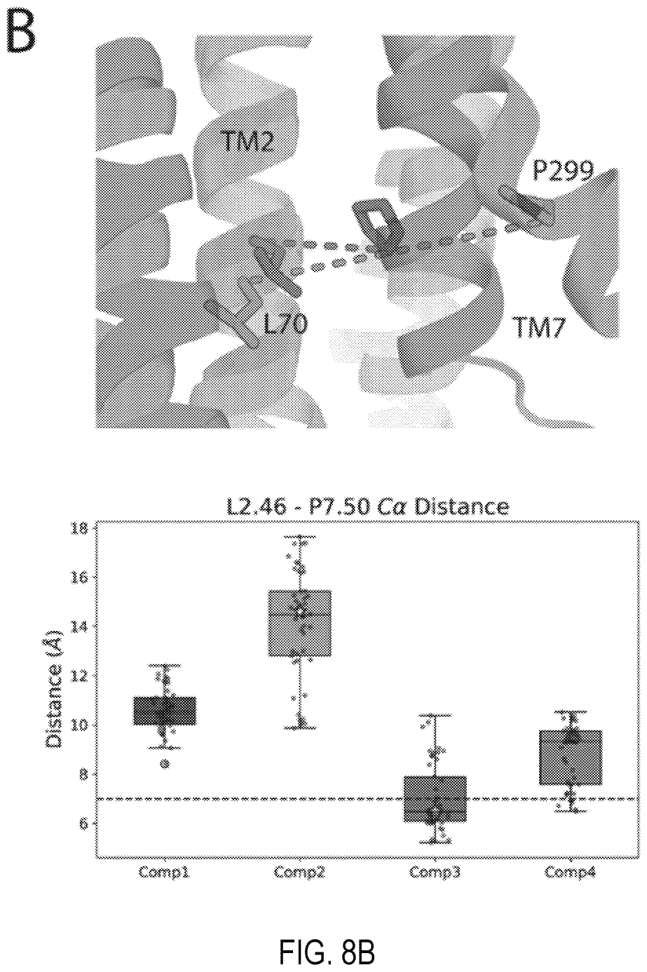

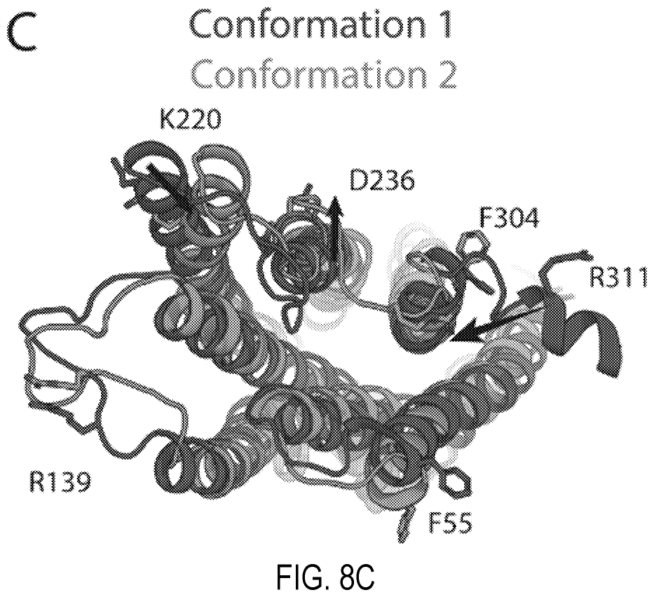

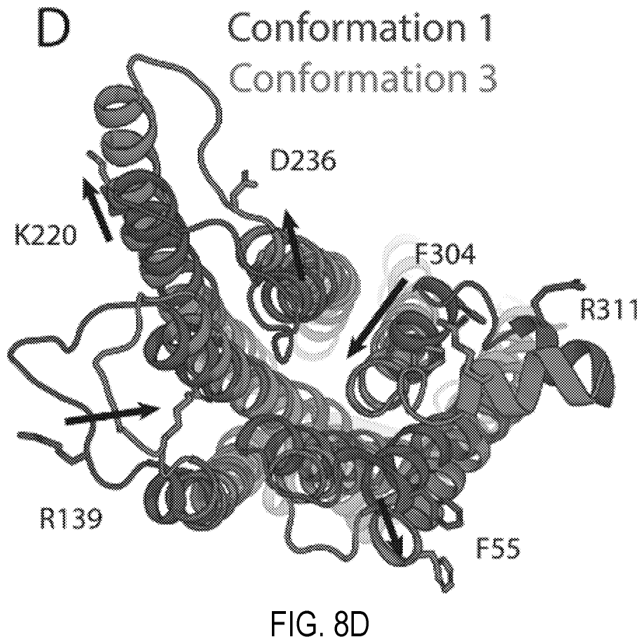

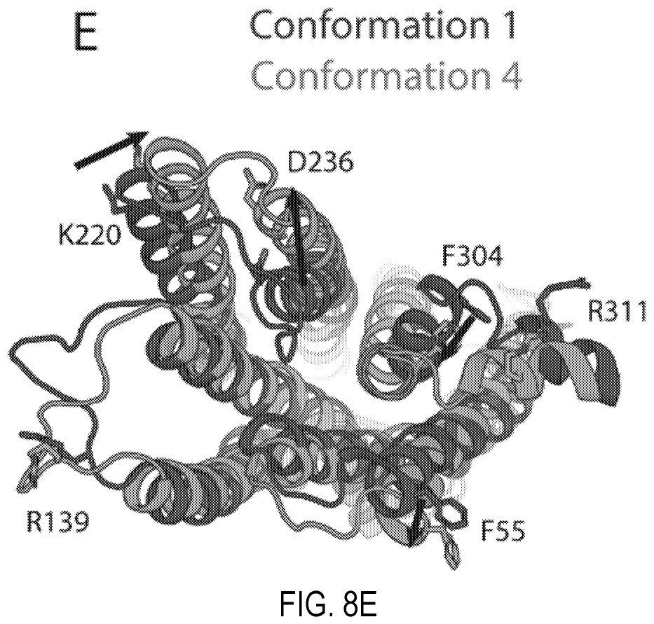

Principle component analysis (PCA) conducted on the coordinates of the protein backbone atoms for the top 50 models generated from each component (FIG. 8A) reveals the non-random pattern of model dispersion—in PCA space—from the same starting model. The results demonstrate that the models undergo global conformational rearrangements from the linear bias derived from distance constraints on a small subset of atoms within the protein system. When projected on to the protein backbone, the origin of PC1 involves most of the cytoplasmic surface of the receptor with inward and outward movements of ICL2, TM1, TM5, TM6, TM7 with respect to the center of the helical bundle, as well as an up and down motion of H8 with respect to the membrane. PC2 origins show much more subtle motions of the cytoplasmic helices; primarily lateral motions in H8 coupled with minor motions of TM6. PCA conducted on the Cα atom coordinates of the individual label sites exhibit a similar pattern to the backbone PCA. As expected, the label site Cα PCA separates into discernable clusters (FIG. 8A), due to bias placed on the system as a function of the distance between label site Cas. The cluster representation similarity between the backbone PCA and label site PCA demonstrates that while the label sites are specifically targeted to meet an experimental constraint, the rest of the system responds to the bias in a coordinated manner.

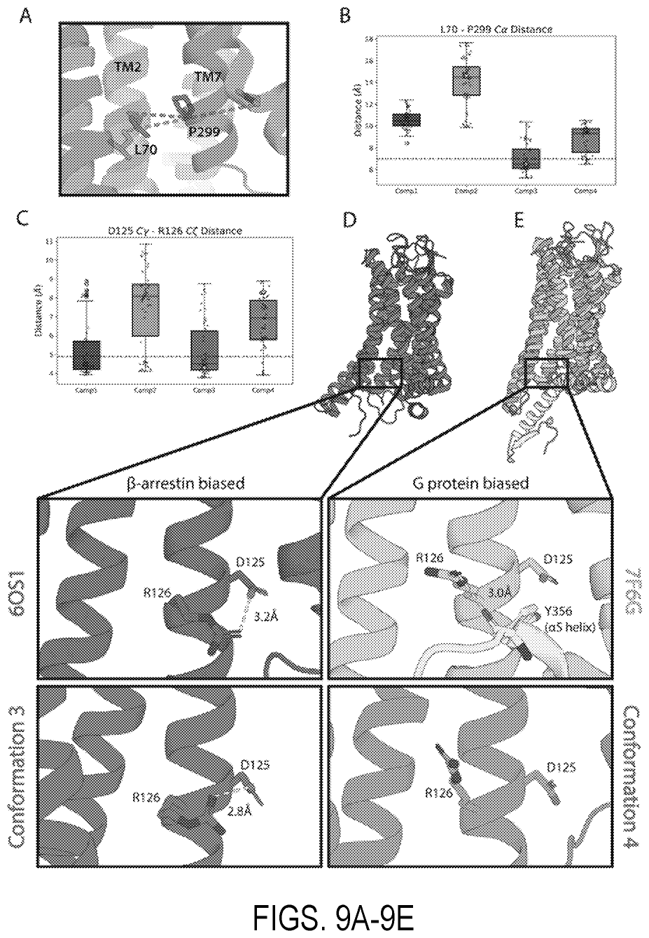

To demonstrate motif-specific conformational rearrangements, we highlight the relatively large movements of the NPxxY motif and more subtle residue rearrangements of the DRY motif, both of which are conserved motifs among class A GPCRs spanning residues N7.49 to Y7.53 (Ballesteros-Weinstein numbering) and D3.49 to Y3.51, respectively. FIG. 9A shows Cα distances between P7.50 of the NPxxY motif and L2.46 (P299 and L70 in the ATIR) of representative models for conformation 2 and conformation 3; a decrease in this distance indicates an inward displacement of TM7 associated with GPCR activation. The L70-P299 Cα distance was measured across all models and plotted (FIG. 9B) and showed statistically significant differences observed between each of the different conformational ensembles (p-value<0.05, Table S2). 68% of the top 50 conformation 3 models fall below the L70-P299 7 Å threshold for β arrestin bias positioning (black dotted line, FIG. 9B). Rearrangements in the residues of the DRY motif were also monitored, specifically the distance between D125 Cγ and R126 Cζ atoms of models generated from each component (FIG. 9C). A distance below 4.9 Å results in D125 oxygen and R126 nitrogen atoms in close proximity to permit either electrostatic interactions or salt bridge formation, forming a “closed configuration” of D125-R126 (black dotted line, FIG. 9C). The β arrestin biased conformation 3 model overlayed with TRV026-bound (β arrestin biased ligand) ATIR crystal structure 6OS1 demonstrates a closed configuration of D125-R126, whereas the Gαq biased conformation 4 model overlayed with ATIR-Gq complex cryo-EM structure demonstrates an “open configuration” of D125-R126. Statistically significant differences are observed across all conformations (p<0.05, Table 3) with the exception of conformations 1 and 3 showing no significant difference. Summarizing the configurations across modeled components, components 1 and 3 both show 66% of models in a closed D125-R126 configuration, whereas components 2 and 4 show 76% and 88% of models in an open D125-R126 configuration FIG. 9C.

TABLE 3
Tukey's HSD test of D125 Cy-R126 Cζ distances between
each generated component ensemble. The top 50 models in
each component were used (displayed in FIG. 8B). The test
statistic, computed for each pairing, is the difference
between the sample means. 95% confidence intervals of the
test statistic are also provided (Lower and Upper CI).

| | | | Lower | Upper |
|---|---|---|---|---|
| Comparison | Statistic | p-value | CI | CI |
| 1-2 | −2.404 | 0.000 | −3.265 | −1.543 |
| 1-3 | −0.008 | 1.000 | −0.869 | 0.853 |
| 1-4 | −1.445 | 0.000 | −2.306 | −0.584 |
| 2-1 | 2.404 | 0.000 | 1.543 | 3.265 |
| 2-3 | 2.396 | 0.000 | 1.535 | 3.257 |
| 2-4 | 0.959 | 0.022 | 0.099 | 1.82 |
| 3-1 | 0.008 | 1.000 | −0.853 | 0.869 |
| 3-2 | −2.396 | 0.000 | −3.257 | −1.535 |
| 3-4 | −1.437 | 0.000 | −2.298 | −0.576 |
| 4-1 | 1.445 | 0.000 | 0.584 | 2.306 |
| 4-2 | −0.959 | 0.022 | −1.82 | −0.099 |
| 4-3 | 1.437 | 0.000 | 0.576 | 2.298 |

Overlays of representative models for conformations 2-4 with a representative model of conformation 1 are shown in FIGS. 8C-8E to highlight the structural differences between conformations. To highlight relevant changes between the component models here, the overlayed representative model Cα change must be >3 Å compared to the starting model for the Cα change of interest. Conformation 2 shows a 4.9 Å inward shift of TM5, a 6.9 Å outward and upward shift of TM6, and a 17.7 Å displacement of H8 compared to component 1 (FIG. 8C). Conformation 3 shows the most differences from component 1 with outward movements of TM1, TM5, and TM6 by 6.9 Å, 3.2 Å, and 6.5 Å, respectively (FIG. 8D). In comparison to conformation 1, the conformation 3 model also shows an inward shift of 9.6 Å in residue 304 (TM7) and a 9.7 Å inward positioning of residue 139 (ICL2) (FIG. 8D). Lastly, conformation 4 shows a 3.9 Å shift of residue 220 (TM5) approaching TM6, while residue 236 (TM6) moves 6.3 Å into an outward position (FIG. 8E). Residue 304 (TM7) adopts an inward position, moving 7.8 Å towards the helical bundle and residue 55 (TM1) accommodates for this by shifting 5.3 Å away from the helical core.

Interestingly, crystal structures of ATIR bound to TRV023 (60S1) and TRV026 (60S2) were observed to be identical in helical arrangement, which is inconsistent with NMF analysis of the DEER data. Component 2 was most highly populated in ATIR bound to TRV026, and component 3 was the dominant component for ATIR bound to TRV023. As described above, conformations 2 and 3 were structurally distinct, yet the crystal structures of ATIR bound to TRV023 and TRV026 did not indicate distinct effects of these ligands at the cytoplasmic surface of the receptor. This illustrates that DEER spectroscopy coupled with this framework can reveal structural effects of ligand binding that are obscured by the use of nanobodies commonly used in cryo-EM and crystallography.

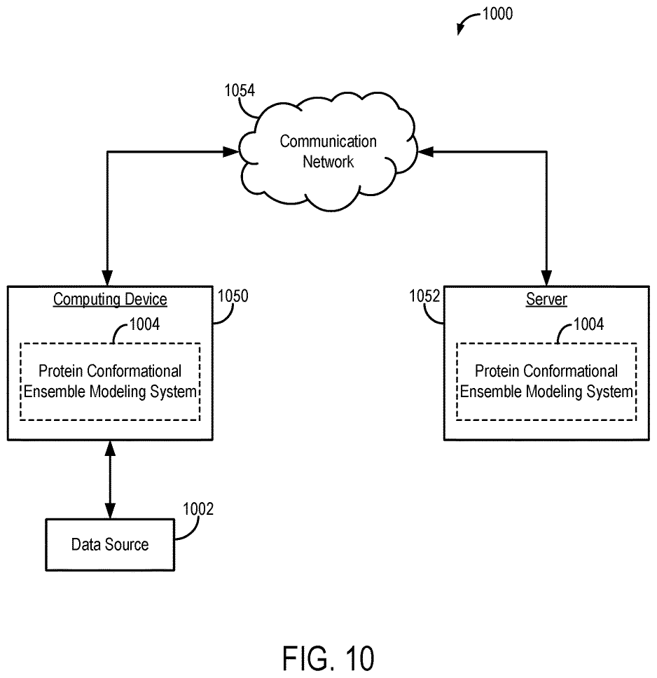

FIG. 10 shows an example of a system 1000 for modeling protein conformational ensembles using distance constraints in accordance with some embodiments described in the present disclosure. As shown in FIG. 10, a computing device 1050 can receive one or more types of data (e.g., EPR spectroscopy data, DEER spectroscopy data, experimental distance distribution data, x-ray crystallography data, cryo-electron microscopy data, nuclear magnetic resonance spectroscopy data, computational protein structure prediction data, etc.) from data source 1002. In some embodiments, computing device 1050 can execute at least a portion of a protein conformational ensemble modeling system 1004 to model protein conformational ensembles using spectroscopic distance constraints based on data received from the data source 1002.

Additionally or alternatively, in some embodiments, the computing device 1050 can communicate information about data received from the data source 1002 to a server 1052 over a communication network 1054, which can execute at least a portion of the protein conformational ensemble modeling system 1004. In such embodiments, the server 1052 can return information to the computing device 1050 (and/or any other suitable computing device) indicative of an output of the protein conformational ensemble modeling system 1004.

In some embodiments, computing device 1050 and/or server 1052 can be any suitable computing device or combination of devices, such as a desktop computer, a laptop computer, a smartphone, a tablet computer, a wearable computer, a server computer, a virtual machine being executed by a physical computing device, and so on. The computing device 1050 and/or server 1052 can also reconstruct images from the data.

In some embodiments, data source 1002 can be any suitable source of data (e.g., measurement data, distance distribution data generated from measurement data, processed measurement data), such as a spectroscopy system, another computing device (e.g., a server storing measurement data, distance distribution data generated from measurement data, processed measurement data), and so on. In some embodiments, data source 1002 can be local to computing device 1050. For example, data source 1002 can be incorporated with computing device 1050 (e.g., computing device 1050 can be configured as part of a device for measuring, recording, estimating, acquiring, or otherwise collecting or storing data). As another example, data source 1002 can be connected to computing device 1050 by a cable, a direct wireless link, and so on. Additionally or alternatively, in some embodiments, data source 1002 can be located locally and/or remotely from computing device 1050, and can communicate data to computing device 1050 (and/or server 1052) via a communication network (e.g., communication network 1054).

In some embodiments, communication network 1054 can be any suitable communication network or combination of communication networks. For example, communication network 1054 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 1054 can be a local area network, a wide area network, a public network (e.g., the Internet), a private or semi-private network (e.g., a corporate or university intranet), any other suitable type of network, or any suitable combination of networks. Communications links shown in FIG. 10 can each be any suitable communications link or combination of communications links, such as wired links, fiber optic links, Wi-Fi links, Bluetooth links, cellular links, and so on.

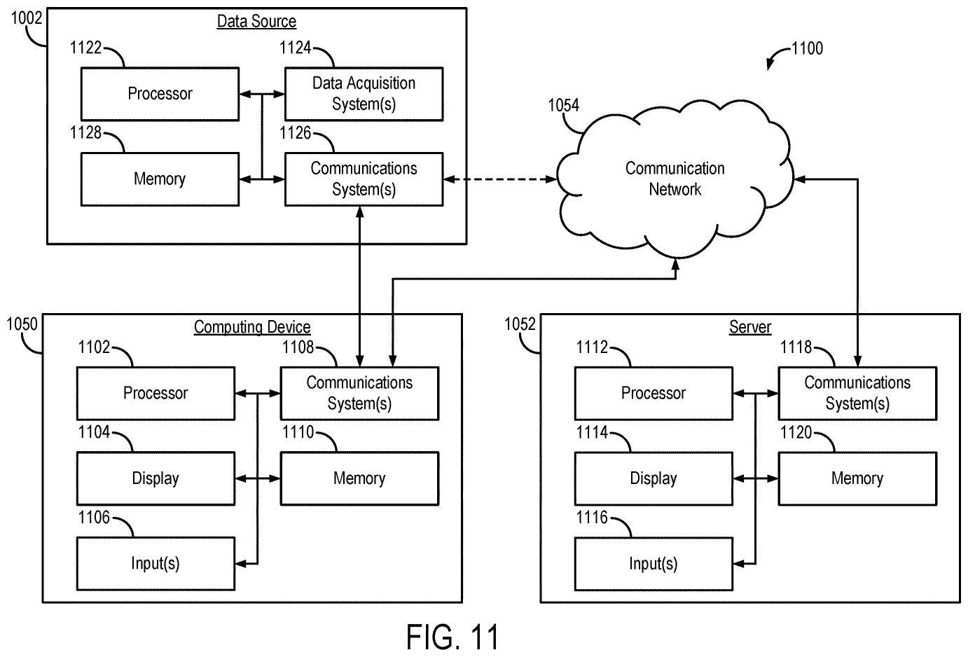

Referring now to FIG. 11, an example of hardware 1100 that can be used to implement data source 1002, computing device 1050, and server 1052 in accordance with some embodiments of the systems and methods described in the present disclosure is shown.

As shown in FIG. 11, in some embodiments, computing device 1050 can include a processor 1102, a display 1104, one or more inputs 1106, one or more communication systems 1108, and/or memory 1110. In some embodiments, processor 1102 can be any suitable hardware processor or combination of processors, such as a central processing unit (CPU), a graphics processing unit (GPU), and so on. In some embodiments, display 1104 can include any suitable display devices, such as a liquid crystal display (LCD) screen, a light-emitting diode (LED) display, an organic LED (OLED) display, an electrophoretic display (e.g., an “e-ink” display), a computer monitor, a touchscreen, a television, and so on. In some embodiments, inputs 1106 can include any suitable input devices and/or sensors that can be used to receive user input, such as a keyboard, a mouse, a touchscreen, a microphone, and so on.

In some embodiments, communications systems 1108 can include any suitable hardware, firmware, and/or software for communicating information over communication network 1054 and/or any other suitable communication networks. For example, communications systems 1108 can include one or more transceivers, one or more communication chips and/or chip sets, and so on. In a more particular example, communications systems 1108 can include hardware, firmware, and/or software that can be used to establish a Wi-Fi connection, a Bluetooth connection, a cellular connection, an Ethernet connection, and so on.

In some embodiments, memory 1110 can include any suitable storage device or devices that can be used to store instructions, values, data, or the like, that can be used, for example, by processor 1102 to present content using display 1104, to communicate with server 1052 via communications system(s) 1108, and so on. Memory 1110 can include any suitable volatile memory, non-volatile memory, storage, or any suitable combination thereof. For example, memory 1110 can include random-access memory (RAM), read-only memory (ROM), electrically programmable ROM (EPROM), electrically erasable ROM (EEPROM), other forms of volatile memory, other forms of non-volatile memory, one or more forms of semi-volatile memory, one or more flash drives, one or more hard disks, one or more solid state drives, one or more optical drives, and so on. In some embodiments, memory 1110 can have encoded thereon, or otherwise stored therein, a computer program for controlling operation of computing device 1050. In such embodiments, processor 1102 can execute at least a portion of the computer program to present content (e.g., images, user interfaces, graphics, tables), receive content from server 1052, transmit information to server 1052, and so on. For example, the processor 1102 and the memory 1110 can be configured to perform the methods described herein (e.g., the workflow illustrated in FIGS. 1A-1C, the method of FIG. 2).

In some embodiments, server 1052 can include a processor 1112, a display 1114, one or more inputs 1116, one or more communications systems 1118, and/or memory 1120. In some embodiments, processor 1112 can be any suitable hardware processor or combination of processors, such as a CPU, a GPU, and so on. In some embodiments, display 1114 can include any suitable display devices, such as an LCD screen, LED display, OLED display, electrophoretic display, a computer monitor, a touchscreen, a television, and so on. In some embodiments, inputs 1116 can include any suitable input devices and/or sensors that can be used to receive user input, such as a keyboard, a mouse, a touchscreen, a microphone, and so on.

In some embodiments, communications systems 1118 can include any suitable hardware, firmware, and/or software for communicating information over communication network 1054 and/or any other suitable communication networks. For example, communications systems 1118 can include one or more transceivers, one or more communication chips and/or chip sets, and so on. In a more particular example, communications systems 1118 can include hardware, firmware, and/or software that can be used to establish a Wi-Fi connection, a Bluetooth connection, a cellular connection, an Ethernet connection, and so on.

In some embodiments, memory 1120 can include any suitable storage device or devices that can be used to store instructions, values, data, or the like, that can be used, for example, by processor 1112 to present content using display 1114, to communicate with one or more computing devices 1050, and so on. Memory 1120 can include any suitable volatile memory, non-volatile memory, storage, or any suitable combination thereof. For example, memory 1120 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1120 can have encoded thereon a server program for controlling operation of server 1052. In such embodiments, processor 1112 can execute at least a portion of the server program to transmit information and/or content (e.g., data, images, a user interface) to one or more computing devices 1050, receive information and/or content from one or more computing devices 1050, receive instructions from one or more devices (e.g., a personal computer, a laptop computer, a tablet computer, a smartphone), and so on.

In some embodiments, the server 1052 is configured to perform the methods described in the present disclosure. For example, the processor 1112 and memory 1120 can be configured to perform the methods described herein (e.g., the workflow illustrated in FIGS. 1A-1C, the method of FIG. 2).

In some embodiments, data source 1002 can include a processor 1122, one or more data acquisition systems 1124, one or more communications systems 1126, and/or memory 1128. In some embodiments, processor 1122 can be any suitable hardware processor or combination of processors, such as a CPU, a GPU, and so on. In some embodiments, the one or more data acquisition systems 1124 are generally configured to acquire data (e.g., spectroscopy data) and can include one or more spectroscopy systems, such as a CW EPR spectroscopy system, a DEER spectroscopy system, or the like. Additionally or alternatively, in some embodiments, the one or more data acquisition systems 1124 can include any suitable hardware, firmware, and/or software for coupling to and/or controlling operations of a spectroscopy system. In some embodiments, one or more portions of the data acquisition system(s) 1124 can be removable and/or replaceable.

Note that, although not shown, data source 1002 can include any suitable inputs and/or outputs. For example, data source 1002 can include input devices and/or sensors that can be used to receive user input, such as a keyboard, a mouse, a touchscreen, a microphone, a trackpad, a trackball, and so on. As another example, data source 1002 can include any suitable display devices, such as an LCD screen, an LED display, an OLED display, an electrophoretic display, a computer monitor, a touchscreen, a television, etc., one or more speakers, and so on.

In some embodiments, communications systems 1126 can include any suitable hardware, firmware, and/or software for communicating information to computing device 1050 (and, in some embodiments, over communication network 1054 and/or any other suitable communication networks). For example, communications systems 1126 can include one or more transceivers, one or more communication chips and/or chip sets, and so on. In a more particular example, communications systems 1126 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1128 can include any suitable storage device or devices that can be used to store instructions, values, data, or the like, that can be used, for example, by processor 1122 to control the one or more data acquisition systems 1124, and/or receive data from the one or more data acquisition systems 1124; to generate images from data; present content (e.g., data, images, a user interface) using a display; communicate with one or more computing devices 1050; and so on. Memory 1128 can include any suitable volatile memory, non-volatile memory, storage, or any suitable combination thereof. For example, memory 1128 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 1128 can have encoded thereon, or otherwise stored therein, a program for controlling operation of data source 1002. In such embodiments, processor 1122 can execute at least a portion of the program to generate images, transmit information and/or content (e.g., data, images, a user interface) to one or more computing devices 1050, receive information and/or content from one or more computing devices 1050, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for modeling protein conformational ensembles using distance constraints, the method comprising:
obtaining experimental distance distribution data for a protein;
generating an initial protein structural model;
iteratively refining the protein structural model by:
simulating a distance distribution for the current model using in silico spin labeling,
comparing the simulated distance distribution to the experimental distance distribution data,
calculating a distance update metric based on differences between the simulated distance distribution and the experimental distance distribution, and
applying a bias potential in a molecular dynamics simulation to drive the protein structure toward target distances updated by the distance update metric; and
generating an ensemble of refined protein structural models consistent with the experimental distance distribution data.

2. The method of claim 1, wherein the distance update metric comprises an update to alpha carbon distances.

3. The method of claim 2, wherein the target distances comprise updated alpha carbon distances.

4. The method of claim 3, wherein calculating the update to the alpha carbon distances comprises:
subtracting the simulated distance distribution from the experimental distance distribution to obtain a residual distribution;
applying a rectified linear unit function to the residual distribution;
calculating a probability-weighted average distance from the resulting positive residual distribution and the simulated distance distribution;
calculating differences between the probability-weighted average distances; and
multiplying the differences by a learning rate parameter to provide an incremental distance update to the alpha carbon distances.

5. The method of claim 4, further comprising applying a momentum parameter to incorporate previous model distributions into the residual distribution calculation.

6. The method of claim 3, wherein applying the bias potential in the molecular dynamics simulation comprises:
determining a linear bias potential to drive the alpha carbon distances to the updated alpha carbon distances;
applying the linear bias potential until the alpha carbon distances converge to the updated alpha carbon distances; and
continuing the simulation for a specified time with the linear bias potential intact.

7. The method of claim 6, wherein the experimental distance distribution data comprises distance distributions for a plurality of spin label pairs, and wherein determining the linear bias potential comprises simultaneously optimizing linear bias potentials for all of the plurality of spin label pairs to enable coordinated conformational rearrangements.

8. The method of claim 1, wherein the experimental distance distribution data are obtained using double electron-electron resonance (DEER) spectroscopy.

9. The method of claim 8, wherein the DEER spectroscopy is performed on a protein labeled with nitroxide spin labels.

10. The method of claim 1, wherein the initial protein structural model is generated using at least one of x-ray crystallography, cryo-electron microscopy, nuclear magnetic resonance spectroscopy, or a computational protein structure prediction.

11. The method of claim 1, wherein simulating the distance distribution using in silico spin labeling is performed using a rotamer library of spin label conformations.

12. The method of claim 1, wherein comparing the simulated distance distribution to the experimental distance distribution data comprises calculating a Jensen-Shannon distance between the distributions.

13. The method of claim 1, wherein generating the ensemble of refined protein structural models comprises:
ranking models based on agreement with the experimental distance distribution data; and
selecting a subset of top-ranked models to form the ensemble.

14. The method of claim 13, wherein ranking the models comprises calculating at least one of a Jensen-Shannon distance or an inverse dot product between the simulated distance distribution for each model and the experimental distance distribution.

15. The method of claim 1, further comprising analyzing the ensemble of refined protein structural models to extract conformational efficacy information for ligands with varied pharmacological efficacy bound to the protein.

16. The method of claim 15, wherein analyzing the ensemble comprises comparing structural rearrangements across different ligand-bound states of the protein.

17. The method of claim 1, wherein the protein is a G protein-coupled receptor.

18. The method of claim 17, wherein the G protein-coupled receptor is one of a β2 adrenergic receptor or an angiotensin II type 1 receptor.

19. The method of claim 1, further comprising filtering the refined protein structural models to exclude models with undesired changes in secondary structure.

20. The method of claim 1, further comprising performing principal component analysis on the ensemble of refined protein structural models to identify conformational changes.
